(19) United States
(12) Patent Application Publication
(10) Pub. No.: US 2011/0060335 A1
(43) Pub. Date: Mar. 10, 2011

(54) Apparatus for Tissue Fusion and Method of Use

(75) Inventors: Jennifer S. Harper, Westminster, CO (US); Barbara R. Bastian, Erie, CO (US)

(73) Assignee: TYCO Healthcare Group LP

(21) Appl. No.: 12/556,796

(22) Filed: Sep. 10, 2009

(51) Int. Cl. A61B 18/14

(52) U.S. Cl. 606/52

(57) ABSTRACT

An end effector for an electrosurgical forceps is provided. The end effector includes a first jaw member having proximal and distal ends and a second jaw member having proximal and distal ends. The distal end of the first jaw member includes a recess defined therein. The distal end of the second jaw member includes a first prong configured for receipt within the recess defined in the first jaw member. Wherein engagement of the first prong of the second jaw member within the recess of the first jaw member creates a gap between the first and second jaw members to enable tissue sealing.

### BACKGROUND

1. Technical Field

The present disclosure relates to an apparatus and method for the fusion of internal tissue and, more particularly, to jaw members including integrally formed stop members.

2. Background of Related Art

Devices for fusing or sealing internal tissue are known. Many of such devices include a pair of jaw members extending from a distal end of a handle assembly. Typically, one or both of the jaw members include an electrode operably connected to an electrosurgical generator. Tissue received between the jaw members are fused through a combination of pressure exerted on the tissue by the jaw members, the gap distance between the jaw members and the electrosurgical energy provided to the electrodes.

In order to effect a proper seal with larger vessels, two predominant mechanical parameters should be accurately controlled—the pressure applied to the vessel and the gap distance between the electrodes—both of which are affected by the thickness of the sealed vessel. With respect to smaller vessels, the pressure applied to the tissue tends to become less relevant whereas the gap distance between the electrically conductive surfaces becomes more significant for effective sealing.

To prevent contact of the electrodes and to set a proper gap for sealing, one or more stop members have been placed on either or both of the jaw members and/or the electrodes. These stop members are composed of ceramic or other suitable insulting material. The addition of a stop member on either or both of the jaw members increases the overall size of the device and/or reduces the size of the sealing surface. As more and more procedures are being performed laparoscopicly, arthroscopicly, endoscopically, and otherwise, there exists a need for smaller sealing devices.

### SUMMARY

Accordingly, an end effector for an electrosurgical forceps is provided. The end effector includes a first jaw member having proximal and distal ends, the distal end including a recess defined therein and a second jaw member having proximal and distal ends. The distal end of the second jaw member including a first prong configured for receipt within the recess defined in the first jaw member. Engagement of the first prong of the second jaw member within the recess of the first jaw member creates a gap between the first and second jaw members and is configured to prevent splaying of the first and second jaw members relative to one another.

In one embodiment, the gap between the first and second jaw members is about 0.001 inches to about 0.006 inches. The first jaw member may include a first electrode and the second jaw member may include a second electrode, the first electrode positioned proximal to the recess and the second electrode positioned proximal to the first prong. The first and second electrodes may be flush relative to respective first and second tissue contacting surface of respective first and second jaw members. The recess in the first jaw member may be defined by a pair of prongs extending from a distal end thereof. The first prong in the second jaw member may include a pair of shoulder portions configured to engage the pair of prongs formed in the first jaw member.

Also provided is a system for the treating of tissue. The system includes an apparatus having first and second jaw members configured to selectively receive tissue therebetween. The first jaw member includes proximal and distal ends, the distal end defining a recess therein. The second jaw member includes proximal and distal ends, the proximal end of the second jaw member pivotably coupled to the proximal end of the first jaw member. The distal end of the second jaw member is in a spaced apart relationship with the first jaw member when in a first position, and the distal end of the second jaw member is received within the recess formed in the first jaw member when in a second position. A gap is formed between the first and second jaw members when in the second position, and a source of electrosurgical energy may be operably connected to at least one of the jaw members to deliver electrosurgical energy to the tissue.

### BRIEF DESCRIPTION OF THE DRAWINGS

The foregoing summary, as well as the following detailed description will be better understood when read in conjunction with the appended figures. For the purpose of illustrating the present disclosure, various embodiments are shown. It is understood, that the present disclosure is not limited to the precise arrangement and instrumentalities shown.

FIG. 1 is a side view of a distal end of forceps jaws according to an embodiment of the present disclosure, in a first or closed position;

FIG. 2 is an end view of the forceps jaws of FIG. 1;

FIG. 3A is a partial, top view of the bottom jaw member of FIGS. 1 and 2;

FIG. 3B is a partial, top view of the top jaw member of FIGS. 1 and 2;

FIG. 4 is a partial, cross-sectional, side view of the jaw members of FIGS. 1-3, shown in a first or closed position, operably connected to an endoscopic device including a source of electrosurgical energy;

FIG. 5 is a partial, cross-sectional, side view of the jaw members of FIG. 4, shown in a second or open position;

FIG. 6 is a partial, side view of jaw members according to an alternate embodiment of the present disclosure, shown in a first or closed position;

FIG. 7 is a cross-sectional side view of the jaw members of FIG. 6;

FIG. 8 is a top view of the bottom jaw member of the jaw members of FIGS. 6 and 7;

FIG. 9 is a partial, cross-sectional, side view of the jaw members of FIGS. 1-3, shown in a first or closed position, operably connected to an alternate embodiment of an endoscopic device; and

FIG. 10 is a partial, cross-sectional, side view of the jaw members of FIG. 9, shown in a second or open position.

### DETAILED DESCRIPTION OF EMBODIMENTS

For the purpose of illustrating the present disclosure, various embodiments are shown. It is understood, however, that the present disclosure is not limited to the precise arrangement and instrumentalities shown. As shown in the drawings and described throughout the following description, as is traditional when referring to relative positioning on an object, the term “proximal” refers to the end of the apparatus that is closer to the user and the term “distal” refers to the end of the apparatus that is further from the user.

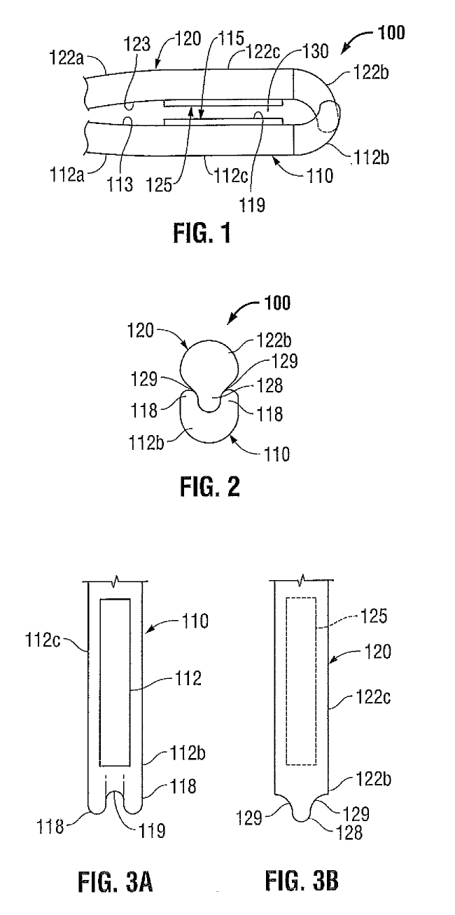

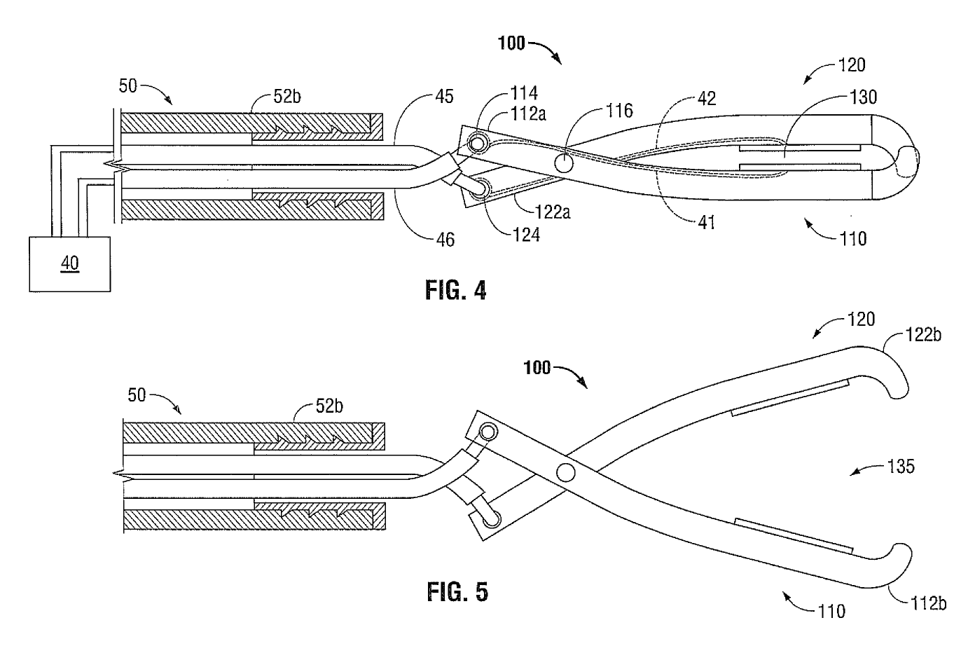

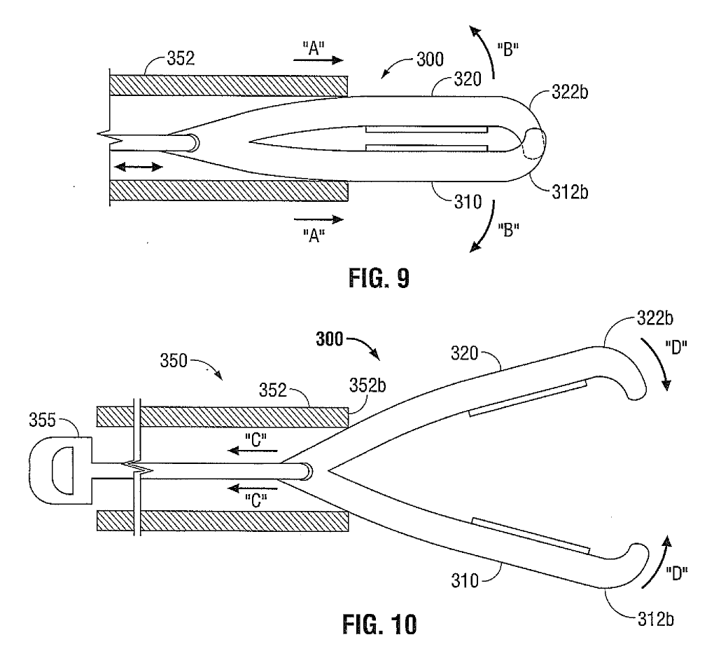

Referring to FIGS. 1-5, an illustrative embodiment of the presently disclosed jaw members are shown and generally designated as end effector assembly 100. End effector assembly 100 includes a first jaw member 110 and a second jaw member 120. In one embodiment, first and second jaw members 110, 120 are pivotably connected to one another by a pivot pin 116 (FIG. 4). Alternatively, first and second jaw members 110, 120 may be integrally formed (FIG. 9) or otherwise connected. Jaw members 110, 120 are configured to retain, seal, cauterize, and/or sever tissue grasped therebetween.

End effector assembly 100 may be incorporated into a hand-held instrument for use in open surgical procedures or may instead be configured, as shown, for incorporation into endoscopic or laparoscopic instruments for use in closed surgical procedures. First and second jaw members 110, 120 may be composed of plastics, polymers, or other insulative material, and combinations thereof. In one embodiment, first and second jaw members 110, 120 may be electrically conductive or have an electrically conductive seal surface. End effector assembly 100 is configured for bipolar operation, as will be discussed below; however, it is envisioned that the aspects of the present disclosure may be modified for monopolar operation.

First and second jaw members 110, 120 form substantially similar semi-arcuate bases having respective proximal ends 112a, 122a, distal ends 112b, 122b and body portions 112c, 122c extending therebetween. First and second jaw members 110, 120 may be of the same or differing widths, and of the same or differing lengths.

Still referring to FIGS. 1-3, in one embodiment, each of first and second jaw members 110, 120 includes an electrode 115, 125, respectively, mounted on body portions 112c, 122c, respectively. Electrodes 115, 125 may be recessed within first and second jaw members 110, 120 such that a top surface thereof is flush with a tissue facing surface 113, 123 of first and second jaw members 110, 120, respectively. Alternatively, and as shown, electrodes 115, 125 may be placed on tissue facing surface 113a, 123a of first and second jaw members 110, 120, respectively. Electrodes 115, 125 may have a flat, curved or textured tissue contacting surface 119, 129. Electrodes 115, 125 are configured for operable connection to a source of electrosurgical energy 40 (FIG. 4). In one embodiment, leads 41, 42 operably connect respective electrodes 115, 125 to generator 40.

Distal ends 112b, 122b of first and second jaw members 110, 120, respectively, are configured to prevent electrodes 115, 125, mounted thereon from contacting one another during tissue sealing. With reference to FIG. 3A, first jaw member 110 includes a pair of prongs 118 forming a curved distal end 112b. Prongs 118 define a curved recess 119 therebetween. Second jaw member 120 includes a prong 128 forming a curved distal end 122b. Prong 128 defines a shoulder portions 129 formed on distal end 122b of second jaw member 120. First and second jaw members 110, 120 are configured such that when distal ends 112b, 122b thereof are approximated towards one another, prong 128 of second jaw member 120 is received within or interlocked with curved recess 119 formed between prongs 118 of first jaw member 110. Prongs 118 of first jaw member 110 are configured to engage shoulder portions 129 of second jaw member 120. In this manner, prongs 118, 128 are configured to maintain body portions 112c, 122e of first and second jaw members 110, 120 in a spaced apart relationship. Thus, a gap 130 is formed between first and second jaw members 110, 120, thereby preventing contact of electrodes 115, 125. Prongs 118 and recess 119 and/or prong 128 and shoulder portions 129 may be configured to provide gap 130 of any size. In this manner, end effector assembly 100 may be configured to seal tissue of various thicknesses. In one embodiment, the gap between first and second jaw members 110, 120 and/or first and second electrodes 115, 125 is from about 0.001 inches to about 0.006 inches. As discussed above, with respect to smaller vessels, the gap distance between the electrodes becomes more significant for effective sealing than the pressure applied to the tissue.

The engagement of prong 128 within recess 119 and the engagement of prongs 118 with shoulders portions 129 cause an interlocking of first and second jaw members 110, 120. This interlocking of distal ends 112b, 122b of first and second jaw members 110, 120, respectively, maintains first and second jaw members 110, 120 in alignment, thereby preventing splaying of jaw members 110, 120 as tissue is captured therebetween.

Proximal ends 112a, 122a of first and second members 110, 120, respectively, may be configured for incorporation into a conventional hand-held forceps or for operable engagement with the distal end of an endoscopic or laparoscopic device. With particular reference to FIGS. 4 and 5, proximal ends 112a, 122a of first and second jaw members 110, 120, respectively define openings 114, 124, respectively, therein, configured for operable engagement with respective actuation cables 45, 46 extending from a distal end 52b of an endoscopic device 50. First and second actuation cables 45, 46 are configured to move first and second jaw members 110, 120. First and second actuation cables 45, 46 may operate in unison or independently of each other to pivot first and/or second jaw members 110, 120 relative to one another. Actuation cables 45, 46 may also be configured to supply electrosurgical energy to first and/or second jaw members 110, 120, respectively, and/or alternatively, to electrodes 115, 125 mounted thereon from a source of electrosurgical energy 40.

Referring to FIG. 5, end effector assembly 100 is shown in a second or open position. In the open position, distal ends 112b, 122b of first and second jaw members 110, 120 are pivoted away from one another to form an opening 135 between first and second jaw members 110, 120. Opening 135 is configured for facilitating the placement of end effector assembly 100 about a portion of tissue, such as, for example the stem of a polyp or a vessel. Depending on the configuration of the actuation mechanism, and whether for open or closed procedures, first jaw member 110 may be held stationary relative to the actuation assembly (not shown) while second jaw member 120 is pivoted about pivot pin 116 relative to first jaw member 110. In an alternate embodiment, both first and second jaw members may be pivoted relative to each other, while in yet another embodiment, second jaw member 120 may be held stationary relative to the actuation assembly while first jaw member 110 is pivoted about pivot pin 116 relative to second jaw member 120. First and second jaw member 110, 120 may be articulated up to and beyond ninety degrees (90°) relative to one another. The range of articulation of second jaw member 120 relative to first jaw member 110 is limited only by the range of motion of the actuation assembly connected thereto.

Turning now to FIGS. 1 and 4, end effector assembly 100 is shown in a first or closed position. In the closed position, gap 130 formed between first and second jaw members 110, 120 is configured for operably retaining the stem of a polyp or other tissue. Whether end effector assembly 100 is of a monopolar or a bipolar design, electrosurgical energy is applied to either first and/or second electrodes 115, 125, at any time during the tissue sealing procedure. For tissue having a greater thickness, it may be necessary to activate the tissue sealing mechanism prior to the complete closure of first and second jaw members 110, 120. As discussed above, the receipt of first prong 128 of second jaw member 120 within recess 119 of first jaw member 110 also prevents the splaying of first and second jaw members 110, 120 as tissue is received and sealed therebetween.

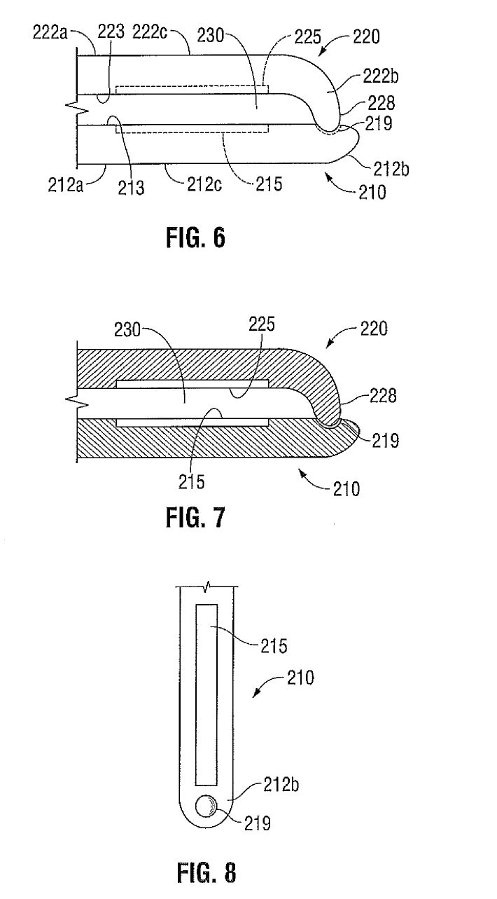

Referring now to FIGS. 6-8, an end effector assembly in accordance with an alternate embodiment of the present disclosure is shown generally as end effector assembly 200. End effector assembly 200 are substantially similar to end effector assembly 100 and will only be described with respect to the difference in construction and operation therebetween. End effector assembly 200 includes first and second jaw members 210, 220. First jaw member 210 forms a level, planar, or linear base including proximal and distal ends 212a, 212b, and a body portion 212c therebetween. Second jaw member 220 includes proximal and distal ends 222a, 222b and a body portion 222c therebetween. As shown, first and second jaw members 210, 220 are integrally connected at proximal ends 212a, 222a, respectively, thereof. In an alternate embodiment, first and second jaw members 210, 220 may be pivotally connected to one another using mechanical fasteners or other suitable affixation methods. Each of first and second jaw members 210, 220 includes an electrode 215, 225, respectively. In the illustrated embodiment, electrodes 215, 225 are recessed within first and second jaw members 210, 220 such that tissue contacting surfaces 213, 223 of electrodes 215, 225, respectively, are flush with respective tissue contacting surfaces 213, 223 of respective first and second jaw members 210, 220.

With continued reference to FIGS. 6-8, a shelf or recess 219 is formed in distal end 212b of first jaw member 210. A tip 228 is formed in distal end 222b of second jaw member 220 and is configured to be received within recess 219 of first jaw member 210 when jaws 200 are in a closed position. The receipt of tip 228 within recess 219 prevents splaying of first and second jaw members 210, 220. The receipt of tip 228 within recess 219 also maintains a gap 230 between first and second jaw members 210, 220 and prevents deflection of second jaw member 220. The width of gap 230 may vary depending on the configuration, i.e. arc, length, tip 228 of second jaw member 220 and/or the depth of recess 219 of first jaw member 210.

With reference now to FIGS. 9 and 10, an alternate embodiment of an actuation mechanism for controlling end effector assembly 300 is shown generally as endoscopic device 350. End effector assembly 300 is substantially similar to end effector assembly 100 described hereinabove. End effector assembly 300 extends from tubular body 352 of endoscopic device 350, in the direction of arrows “A”, causing first and second jaw members 310, 320 to approximate away from each other, in the direction of arrows “B”. Alternatively, tubular body 352 may be retracted relative to end effector assembly 300 to permit the opening of end effector assembly 300. End effector assembly 300 is next positioned about a portion of tissue (not shown) to be sealed. Retraction of a handle 355 of endoscopic instrument 350, in the direction of arrows “C”, causes end effector assembly 300 to engage a distal end 352b of tubular body 352. Continued retraction of handle 355 causes distal ends 312b, 322b of first and second jaw members 310, 320, respectively, to approximate towards each other, in the direction of arrows “D”, until distal end 322h of second jaw member 320 engages distal end 312b of first jaw member 310.

Thus, it should be understood that various changes in form, detail and operation of the tissue removal jaws of the present disclosure may be made without departing from the spirit and scope of the present disclosure.

What is claimed is:

1. An end effector for an electrosurgical forceps, comprising:
a first jaw member having proximal and distal ends, the distal end including a recess defined therein; and
a second jaw member having proximal and distal ends, the distal end including a first prong configured for receipt within the recess defined in the first jaw member, wherein engagement of the first prong of the second jaw member within the recess of the first jaw member creates a gap between the first and second jaw members and is configured to prevent splaying of the first and second jaw members relative to one another.

2. The end effector of claim 1, wherein the gap is about 0.001 inches to about 0.006 inches.

3. The end effector of claim 1, wherein the first jaw member includes a first electrode and the second jaw member includes a second electrode, the first electrode positioned proximal to the recess and the second electrode positioned proximal to the first prong.

4. The end effector of claim 3, wherein the first and second electrodes are flush relative to respective first and second tissue contacting surface of respective first and second jaw members.

5. The apparatus of claim 1, wherein the recess in the first jaw member is defined by a pair of prongs extending from a distal end thereof.

6. The apparatus of claim 5, wherein the first prong in the second jaw member includes a pair of shoulder portions configured to engage the pair of prongs formed in the first jaw member.

7. A system for the treating of tissue, the system comprising:
an apparatus having first and second jaw members configured to selectively receive tissue therebetween,
wherein the first jaw member includes proximal and distal ends, the distal end defining a recess therein, and
wherein the second jaw member includes proximal and distal ends, the proximal end of the second jaw member pivotably coupled to the proximal end of the first jaw member, wherein the distal end of the second jaw member is in a spaced apart relationship with the first jaw member when in a first position, and the distal end of the second jaw member is received within the recess formed in the first jaw member when in a second position, forming a gap between the first and second jaw members; and
a source of electrosurgical energy operably connected to at least one of the jaw members to deliver electrosurgical energy to the tissue.
